(19) United States
(12) Patent Application Publication
(10) Pub. No.: US 2017/0340598 A1
(43) Pub. Date: Nov. 30, 2017

(54) Method of Reducing Uric Acid with Fucoxanthin-Containing Composition

(71) Applicant: TCI Co., Ltd., Taipei (TW)

(72) Inventors: Ling-Fang Zhang, Taipei (TW); Hsiang-Ling Su, Taipei (TW); Yung-Hsiang Lin, Taipei (TW)

(21) Appl. No.: 15/485,450

(22) Filed: Apr. 12, 2017

(30) Foreign Application Priority Data
May 24, 2016 (TW) 105116114

(51) Int. Cl. A61K 31/336; A61K 9/00; A23L 33/105; A61K 36/03

(52) U.S. Cl. CPC A61K 31/336; A61K 36/03; A61K 9/0056; A23L 33/105; A23V 2002/00; A61K 2236/17; A61K 2236/331; A61K 2236/53

(57) ABSTRACT

The present invention provides a method of reducing uric acid in a subject in need thereof, comprising administering to the subject a composition comprising an effective amount of fucoxanthin, wherein the fucoxanthin has the effect of reducing uric acid and preventing gout.

### FIELD OF THE DISCLOSURE

The present invention relates to a method of reducing uric acid in a subject in need thereof, and more particularly, to a method of using a composition comprising fucoxanthin for reducing uric acid and preventing gout.

### BACKGROUND

Gout is characterized by the deposition of uric acid crystals in the joints, such as toes, ankles, knees, wrist joints, fingers, elbows and so on, which causes arthritis in those regions. Clinical diagnosis of gout is defined as urate concentration in the blood of more than 6.8 mg/dl, known as hyperuricemia. When urate concentration in the blood exceeds the renal metabolic threshold, the excess urate forms crystals, or so-called tophi, which accumulate in the joint. The accumulated tophi can cause acute arthritis and inflammation in the joints and in periarticular regions, including the activation of complements and release of inflammatory cytokines. For example, purines that are easily absorbed from beer can promote excessive formation of uric acid. However, research has also shown that the causes of gout may be due to multiple factors, such as genetics, large consumption of alcohol, decrease of the glomerular filtration rate, drugs (such as diuretics, aspirin, and niacin), and so on.

Gout is a common metabolic disease, and hyperuricemia is considered to be the major cause of gout attacks. Statistics show that the prevalence of hyperuricemia in Taiwanese aborigines may be up to 50%, which is attributed to dietary habits, genetics and environmental causes. Allopurinol, a drug used clinically to inhibit uric acid formation, can inhibit xanthine oxidase activity, thereby interfering with the conversion of the hypoxanthine to xanthine and also xanthine to uric acid. This reduces the concentration of uric acid in blood and urine to reduce the likelihood of a gout attack. However, medication compliance with allopurinol is poor because of its side effects such as anemia, nausea, pain, itching, etc. Therefore, finding a natural substance with a uric acid reducing effect to replace the drugs in current clinical use that have multiple side effects is the main focus of researchers in the field.

### SUMMARY

An objective of the present invention is to provide a method of using a composition comprising fucoxanthin for reducing uric acid in a subject in need thereof, which comprising administering to the subject a composition comprising an effective amount of fucoxanthin, wherein the fucoxanthin is obtained by the extraction of seaweed with water at a predetermined temperature for a period of time.

In one embodiment of the present invention, the predetermined temperature is 50□ to 100□, the period of time is 30 to 120 minutes, and the extraction of seaweed with water is conducted at a solid:liquid ratio of 1:5 to 1:20.

In one embodiment of the present invention, the seaweed is dried seaweed, and the seaweed is Ascophyllum nodosum.

In one embodiment of the present invention, the fucoxanthin can inhibit xanthine oxidase activity to reduce uric acid, and the composition is a pharmaceutical composition and/or a food composition.

Through the technical features of the present invention, fucoxanthin provided an excellent ability to inhibit xanthine oxidase activity, and can effectively reduce uric acid to further reduce the risk of gout. The fucoxanthin of the present invention can be used as a health material for reducing uric acid or alleviating gout in general foods or health foods.

The embodiments of the present invention will be further illustrated with the following drawings. The following examples are cited to illustrate the present invention and are not intended to limit the scope of the present invention. Modifications and amendments can be made by persons skilled in the art without departing from the spirit and scope of the present invention. It is intended that the scope of the invention be defined by the claims appended hereto.

### BRIEF DESCRIPTION OF THE DRAWINGS

FIG. 1 is a graph showing the fucoxanthin content of the Ascophyllum nodosum extract of the present invention.

FIG. 2 is a graph showing the effect of the Ascophyllum nodosum extract of the present invention in inhibiting xanthine oxidase activity.

FIG. 3 shows the results of co-treatment of ADTC5 cells with monosodium urate and Ascophyllum nodosum extract for 24 hours.

FIG. 4 shows the results of co-treatment of ADTC5 cells with monosodium urate and Ascophyllum nodosum extract for 48 hours.

### DETAILED DESCRIPTION

Values used herein are approximations and all experimental data values are expressed in the range of 20%, preferably in the range of 10%, and most preferably in the range of 5%.

The present invention provides a method of using a composition comprising fucoxanthin for reducing uric acid in a subject in need thereof, wherein the fucoxanthin is obtained by the extraction of seaweed with water at a predetermined temperature for a period of time, and the seaweed is preferably Ascophyllum nodosum. The molecular formula of the fucoxanthin is C₄₂H₅₈O₆, and its structural formula is shown in formula (I) below.

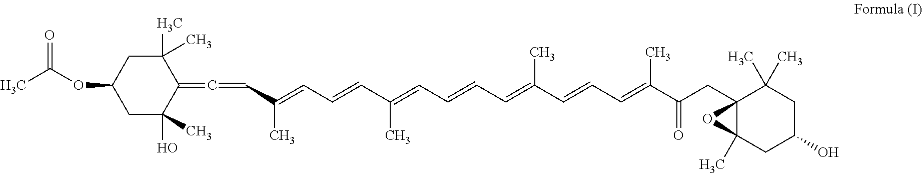

In one embodiment of the present invention, the seaweed is preferably dried seaweed, the predetermined temperature is 50□ to 100□, the period of time is 30 to 120 minutes, and the extraction of seaweed with water is conducted at a solid: liquid ratio of 1:5 to 1:20.

Fucoxanthin, the special compound with uric acid-reducing effect in seaweed, is extracted from seaweed, preferably Ascophyllum nodosum, and purified using extraction technology in the present invention. The uric acid-reducing effect of the extract comprising fucoxanthin was evaluated in terms of its inhibitory efficiency against xanthine oxidase activity. It was further confirmed that the extract comprising fucoxanthin can be used as a health material for reducing uric acid or alleviating gout in general foods or health foods. The present invention will be described in detail by the following Examples 1 to 4.

### EXAMPLE 1

### Seaweed Extraction

The seaweeds used in the present invention include, but are not limited to, Ascophyllum nodosum, Dictyota divaricata, Sargassum cristaefolium, Laminaria japonica, and Hincksia mitchellae. Fresh Ascophyllum nodosum was adopted here and dried for use in the subsequent extraction process. The extraction of dried Ascophyllum nodosum with water was conducted at a solid:liquid ratio of 1:5 to 1:20, at 50□ to 100□, respectively, for 30 to 120 minutes; the mixture was then cooled to room temperature and then filtrated with a 200 μm mesh to obtain Ascophyllum nodosum extract.

In the following Examples, the Ascophyllum nodosum extract will be used to evaluate its xanthine oxidase activity inhibition ability and determine its fucoxanthin content.

### EXAMPLE 2

### Determination of the Fucoxanthin Content

The Ascophyllum nodosum extract obtained from Example 1 was filtered through a 0.45 μm filter, and the content of fucoxanthin was analyzed by HPLC. The analysis conditions are shown as follows:
Instruments: Waters e2695 & Waters 2998 Photodiode Array Detector
Column: Inertsil ODS-3V (5 μm 4.6×250 mm)+guard column
Flow rate: 0.7 ml/min
Injection volume: 20 μL
Column temperature: 35° C.
Detection wavelength: 445 nm
Mobile phase: 75% acetonitrile
Time: 40 minutes
Standard: fucoxanthin

Analysis was performed by drawing a standard curve according to the fucoxanthin standard, then calculating the fucoxanthin content in test sample by interpolation.

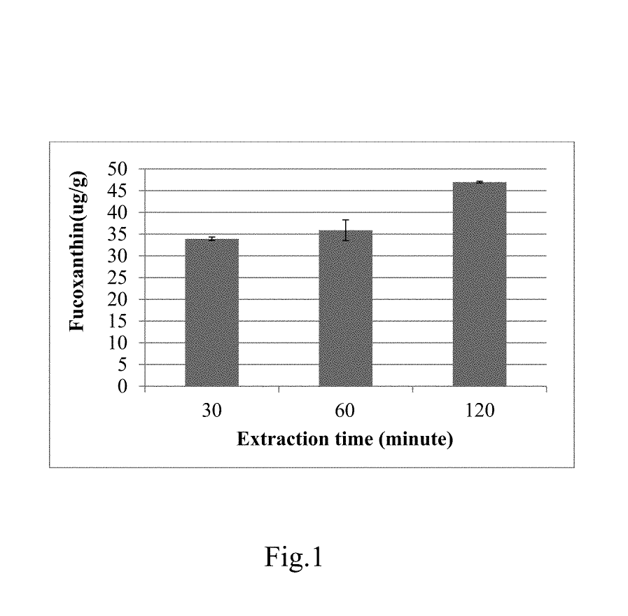

As shown in FIG. 1, with the increase in extraction time, the content of fucoxanthin increases significantly. This shows that the longer the extraction time, the more fucoxanthin from Ascophyllum nodosum that can be extracted by the present method. Therefore, longer extraction times can effectively increase the fucoxanthin content in the extracts.

### EXAMPLE 3

### Evaluation of the Inhibition of XANTHINE OXIDASE ACTIVITY

50 μL of the test sample, 35 μL of phosphate buffer (70 mM, pH 7.5) and 30 μL of xanthine oxidase (0.02 units/mL) were mixed and shaken, and allowed to stand at room temperature for 15 minutes. Then, 60 μL of xanthine (150 μM) was added and allowed to react at room temperature for 30 minutes; the reaction was then stopped by the addition of 100 μL of the HCl (1N), followed by measuring absorbance at 290 nm. The inhibitory effect of the test sample against xanthine oxidase activity was evaluated and compared with the control group.

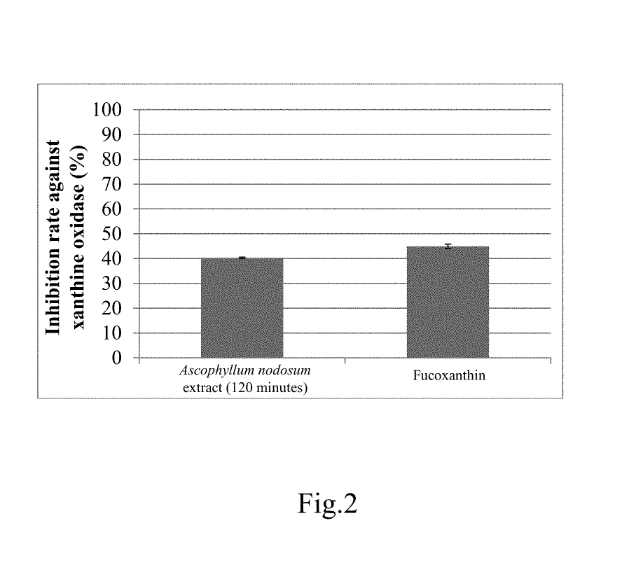

Evaluation results are shown in FIG. 2. Ascophyllum nodosum extract being extracted for 120 minutes showed 40% inhibition rate against xanthine oxidase. The fucoxanthin with the same concentration of the Ascophyllum nodosum extract showed 44% inhibition rate against xanthine oxidase, indicating that inhibitory effect of the Ascophyllum nodosum extract against xanthine oxidase is derived from fucoxanthin.

### EXAMPLE 4

### Cell Experiment

ADTC5 mouse cartilaginous cells (Mouse 129 teratocarcinoma AT805 derived) were seeded into a culture plate at a density of 5×10⁵ cells/well and cultured at 37□ under 5% CO₂ for 24 hours. The culture medium was DMEM/F12 supplemented with 5% fetal bovine serum (FBS) and 1% penicillin-streptomycin (Gibco). Next, the cells were treated with monosodium urate (MSU) (0.125 mg/ml, in medium) to induce uric acid crystallization. Cells were divided into three groups: Group 1 was treated with Ascophyllum nodosum extract extracted for 120 minutes (2 mg/ml, in medium); group 2 was treated with fucoxanthin (1 ppm, in medium); group 3 was untreated (the control group). After 24 and 48 hours of treatment, the cells were harvested and lysed, then washed once with PBS, and the supernatant was removed and 350 μl of the RNA lysate was added for a further RNA extraction process (using GeneMark's RNA purification kit, operated according to the manufacturer's instructions). Next, reverse transcription was performed using a SuperScript™ reverse transcription kit (Invitrogen). The NOS2 (inducible nitric oxide synthase) gene was quantified using the ABI StepOnePlus™ System (Applied Biosystems), and β-actin was used as a reference gene for normalizing each sample. It is known that accumulation of uric acid crystals in the joints causes inflammation in joint cavities, which leads to the increase of NOS2 gene expression in chondrocytes. Therefore, NOS2 gene expression levels were used to evaluate the degree of inflammation of cells caused by uric acid crystallization. The relative quantification of gene expression was performed using the 2^{-ΔΔct} method. Statistical analysis was performed by Student's t-test (Microsoft Excel software).

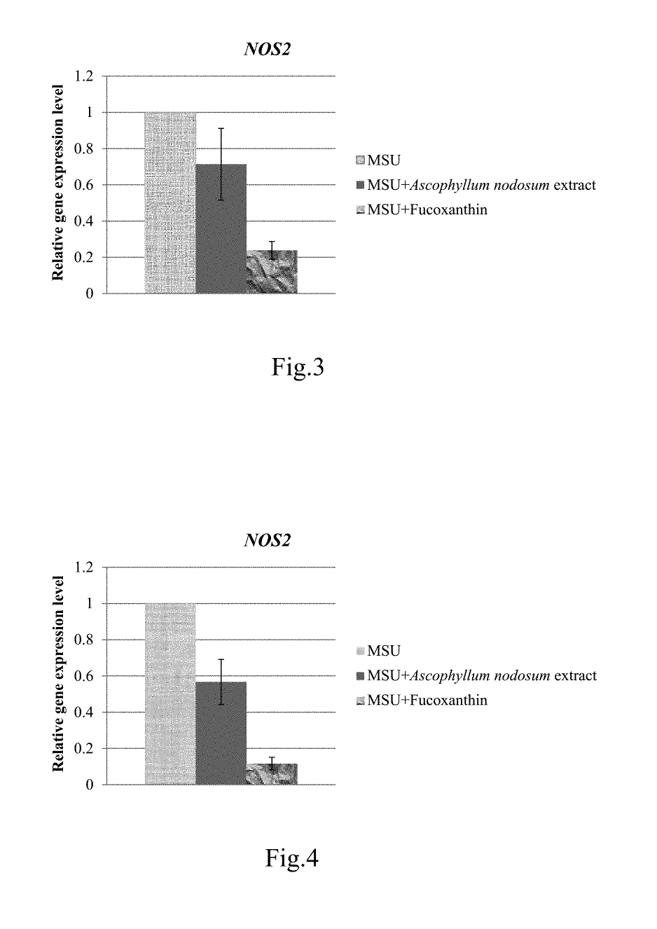

FIG. 3 shows the results of ADTC5 cells co-treated with MSU and Ascophyllum nodosum extract for 24 hours. Ascophyllum nodosum extract can reduce the NOS2 gene expression level in ATDC5 cells. The NOS2 gene expression level in the MSU-induced group was expressed as 1, then the NOS2 gene expression levels in the Ascophyllum nodosum extract-treated group and the fucoxanthin-treated group could be reduced to 0.71 and 0.24, respectively. The results indicate that Ascophyllum nodosum extract can alleviate the inflammation of the joint cavity induced by MSU in the joints. Also, the effect of fucoxanthin can be significantly greater than that of Ascophyllum nodosum extract, indicating that the anti-inflammatory effect ofAscophyllum nodosum extract may come from fucoxanthin.

FIG. 4 shows the results of ADTC5 cells co-treated with MSU and Ascophyllum nodosum extract for 48 hours. Ascophyllum nodosum extract can reduce the NOS2 gene expression level in ATDC5 cells. The NOS2 gene expression level in the MSU-induced group was expressed as 1, then the NOS2 gene expression levels in the Ascophyllum nodosum extract-treated group and the fucoxanthin-treated group could be reduced to 0.57 and 0.12, respectively. The results indicate that Ascophyllum nodosum extract can alleviate the inflammation of the joint cavity induced by MSU in the joints. Also, the effect of fucoxanthin can be significantly greater than that of Ascophyllum nodosum extract, indicating that the anti-inflammatory effect of Ascophyllum nodosum extract may come from fucoxanthin. The results also show that the effect to NOS2 gene by the co-treatment of MSU and Ascophyllum nodosum extract in ATDC5 cells for 48 hours was better than that for 24 hours, indicating that the NOS2 gene inhibitory effect resulted from Ascophyllum nodosum extract and fucoxanthin are long-lasting.

From the above Examples, it can be seen that the fucoxanthin described in the present invention does provide the effect of reducing uric acid. In particular, because the Ascophyllum nodosum extract (extracted for 120 minutes) in the preferred embodiment of the present invention was rich in fucoxanthin, it could be used as a health material for reducing uric acid or alleviating gout in general foods or health foods.

What is claimed is:

1. A method of reducing uric acid in a subject in need thereof, comprising administering to the subject a composition comprising an effective amount of fucoxanthin.

2. The method as claimed in claim 1, wherein the fucoxanthin is obtained by the extraction of seaweed with water at a predetermined temperature for a period of time.

3. The method as claimed in claim 2, wherein the predetermined temperature is 50□ to 100□.

4. The method as claimed in claim 2, wherein the period of time is 30 minutes to 120 minutes.

5. The method as claimed in claim 2, wherein the extraction of seaweed with water is conducted at a solid:liquid ratio of 1:5 to 1:20.

6. The method as claimed in claim 2, wherein the seaweed is Ascophyllum nodosum.

7. The method as claimed in claim 3, wherein the seaweed is Ascophyllum nodosum.

8. The method as claimed in claim 4, wherein the seaweed is Ascophyllum nodosum.

9. The method as claimed in claim 5, wherein the seaweed is Ascophyllum nodosum.

10. The method as claimed in claim 2, wherein the fucoxanthin inhibits xanthine oxidase activity to reduce uric acid.

11. The method as claimed in claim 3, wherein the fucoxanthin inhibits xanthine oxidase activity to reduce uric acid.

12. The method as claimed in claim 4, wherein the fucoxanthin inhibits xanthine oxidase activity to reduce uric acid.

13. The method as claimed in claim 5, wherein the fucoxanthin inhibits xanthine oxidase activity to reduce uric acid.

14. The method as claimed in claim 1, wherein the composition is a pharmaceutical composition and/or a food composition.
